(19) United States
(12) Patent Application Publication
(10) Pub. No.: US 2012/0179004 A1
(43) Pub. Date: Jul. 12, 2012

(54) Medical monitoring network

(76) Inventors: Bernd Roesicke, Mannheim (DE); Sybille Froech, Mannheim (DE); Christian Niesporek, Heidelberg (DE); Wolfgang Reiser, Mannheim (DE)

(21) Appl. No.: 13/177,858

(22) Filed: Jul. 7, 2011

Related U.S. Application Data
(63) Continuation of application No. PCT/EP2010/000116, filed on Jan. 13, 2010.

(30) Foreign Application Priority Data
Jan. 14, 2009 (EP) 09150545.3

(51) Int. Cl. A61B 5/0205; A61B 5/021; A61B 5/1455; A61B 5/024; A61B 5/05; A61B 5/01; A61B 5/145; A61F 2/04; A61M 1/00; A61B 5/00; A61B 5/11

(52) U.S. Cl. 600/301; 600/300; 600/309; 600/485; 600/323; 600/508; 600/595; 600/549; 600/483; 600/324; 600/365; 600/364; 600/345

(57) ABSTRACT

A network for monitoring bodily functions of a patient is disclosed. The network comprises at least two distinct network nodes that can be connected to a body of the patient. At least two of the network nodes can have at least one medical function, such as, for example, a diagnostic function and/or a medication function. The network nodes can communicate directly with one another via the body of the patient and can interchange data and/or commands.

### CROSS-REFERENCE TO RELATED APPLICATIONS

This application is a continuation of PCT/EP2010/000116, filed Jan. 13, 2010, which is based on and claims priority to EP 09 150 545.3, filed Jan. 14, 2000, which is hereby incorporated by reference.

### BACKGROUND

The present disclosure generally relates to a network, a network node and system for monitoring bodily functions of a patient and, in particular, relates to a network for monitoring bodily functions of a patient, a network node for use in such a network, and a medical system that is able to link-in a network to care for patients with chronic diseases and/or risk patients who have a number of bodily functions to be monitored and/or influenced at the same time.

Both clinical settings and private healthcare arrangements often require systems that are able to monitor the complex interactions between individual bodily functions of a patient and, if need be, to influence bodily functions in a targeted fashion. By way of example, this can be the care of chronically ill patients, such as, for example, diabetes patients. Risk patients, those patients who are known to have an increased risk of infarction, can also be cared for in this way. However, the term, “patient,” does not necessarily restrict the target group to ill human or animal patients; rather, in principle, healthy target groups may also be cared for with the devices proposed in this disclosure. Therefore, the term, “patient,” can generally be considered to be synonymous with the term, “user.”

The communication between individual components of a system can often be a challenge, especially with complex medical systems. Radio systems are mainly used these days for wireless communication in the close vicinity of patients. These systems typically utilize the entire electromagnetic field and usually operate in the far-field. In far-field communication, the distance of a receiver from a transmitter antenna is greater than twice the wavelength of the selected wireless carrier frequency. For example, in the case of 2.45 GHz, this is approximately 0.3 m. Various wireless technologies are standardized by IEEE 802.11 and related standards. When an industry-science-medical frequency (ISM frequency, for example 2.45 GHz) is utilized, distances can be between approximately 1 and 10 m and are bridged with a restricted transmission power of, for example, approximately 100 mW. ISM frequencies are generally accessible frequency bands, i.e., they are not assigned according to strict regulations by organizations or governments. The 2.45 GHz band is currently the only ISM frequency band that, if the applicable standards are complied with, can be used world-wide without restrictions.

Systems that only utilize the magnetic field component can be used. For physical reasons, this type of system can only bridge distances within the near-field of the antenna. Such systems can be radiofrequency identification (RFID) systems, also referred to as transponders, or as near field communication (NFC) systems. RFID systems can be distinguished by a reader that induces data and energy into a transponder. The transponder modifies this data where necessary and returns it to the reader. The transponder is generally only active if it is within the field of influence of the energy of the reader. NFC generally operates using the same structures and protocols as RFID. However, with NFC, the transponder also has its own energy source and so that communication is activated by the reader but the application may also remain active outside the influence of the reader. This can be particularly advantageous in the case of distributed, continuously measuring sensor systems.

Additionally, communication systems that only utilize the electric field component of the electromagnetic field have also be known for quite some time now. As a result of the dielectric strength of air, which is approximately 1000 V/mm, the electric field component can transmit at most only approximately 1/90,000 of the energy of the magnetic field. The long-distance-effect component is therefore restricted to direct contact in many cases. However, it was discovered that the human body is relatively well-suited to conducting dielectric displacement currents. Hence, information can be transmitted without large-scale departure from the conducting body. Such networks, which operate in the near-field region and utilize the human body for transmitting signals, are known as personal information and communication and are also referred to as personal area networks (PAN). These networks use electric fields as the communication medium between transmitters that are arranged on the human body.

The medical field also has systems that utilize the human body for transmitting signals, such as, for example, the medical long-term monitoring of a patient, for example, an astronaut. An autonomous sensor unit comprising of electrodes can be arranged on the body of a human. These electrodes are arranged on the skin by adhesive tape. A body-worn transmitter and receiver acting as a central unit is provided.

The communication systems known from the prior art have a number of disadvantages or technical challenges. For example, in the case of far-field communication, transmission energy, and hence the modulated information, is scattered widely in space limiting the transmission bandwidth by the presence of other parties in the same frequency band (e.g., ISM). The presence of many parties in the same frequency band requires complex protocols to secure the transmission of the data. Thus, on the one hand, data integrity has to be ensured and, on the other hand, correct assignment of the data, i.e., to the correct transmitter and/or receiver, also has to be ensured. Furthermore, deliberate data misuse has to be prevented. These measures overall reduce the transmission of useful data per unit time. Since the specified radio systems are used in an increasing number of applications, increased band assignment and a further restriction of the bandwidth to ISM frequencies is to be expected in future. Separate frequency bands, which so far have only been reserved for a very restricted field, are assigned for the field of life-sustaining diagnosis, for example the wireless medical telemetry service (WTMS) frequency range between 402 and 406 MHz for intensive care units in clinics or ambulances. However, there may soon be critical latency times in the transmission of diagnostically relevant data, or therapeutic instructions, which are not in the life-sustaining field, in the case of wireless transmission such as in the case of the ISM bands. Under certain circumstances, this could be relevant to a coupled glucose-insulin system according to the “closed-loop” principle.

As a result of the virtually spherical emission of the transmission energy in far-field communication, both transmission and reception requires a significant amount of energy because the transmitter must always be set to maximum transmission power and the receiver must always be set to maximum reception sensitivity. Both require a significant amount of energy. By contrast, directed emission does not help in corresponding applications because the location of the potential receivers is unknown. Hence, a multiple of the required energy is emitted into space, at least whilst a partner is sought and while the contact is established. Such a waste of energy is generally not permitted, at least in the case of implanted systems.

Nor are free-field transmissions with implanted transmitters generally possible at higher frequencies. In the case of an implanted sensor, a transmission frequency of 2.45 GHz would be largely absorbed by the tissue fluid because, for example, the absorption maximum of water lies at 2.4 GHz. However, suitable low-frequency systems are limited in respect to their application as a result of the required large antenna dimensions and low transmission bandwidth. By way of example, animal identification systems are designed for a relatively low data rate at 125 kHz.

Therefore, it is an aspect of the present invention to provide devices for monitoring bodily functions of a patient by registering measurement data quickly and reliably, in order to be able, as autonomously as possible, to react to critical states.

### SUMMARY

According to the present disclosure, a network for monitoring bodily functions of a patient is disclosed. The network comprises at least two network nodes connected to a body of the patient. Each of the at least two network nodes has at least one medical function. The at least two network nodes communicate directly with one another via the body of the patient and interchange data and/or commands. The at least two network nodes control the network.

In accordance with one embodiment of the present disclosure, different networking principles, or networking technologies for a network close to the body, that is a network on the body, in the body, or in close spatial vicinity of the body can be used.

Accordingly, it is a feature of the embodiments of the present disclosure to provide devices for monitoring bodily functions of a patient by registering measurement data quickly and reliably in order to be able, as autonomously as possible, to react to critical states. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a schematic diagram of a non-ground-related near-field intra-body communication according to an embodiment of the present disclosure.

FIG. 2 illustrates an exemplary embodiment of an intra-body network in the field of diabetes according to an embodiment of the present disclosure.

FIG. 3 illustrates a basic layout for extracting a signal and operational energy from the same source according to an embodiment of the present disclosure.

FIG. 4 illustrates a basic layout for extracting energy from a separate source according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

A network for monitoring bodily functions of a patient is disclosed. The patient need not necessarily be an ill human or an ill animal; rather, healthy patients may also be monitored. In general, monitoring should be understood to mean registering body states, for example physiological body states and/or other body states, which can also, alternatively or additionally, comprise therapeutic steps, i.e., intervening and/or regulating steps, in addition to purely registering these body states and/or collecting and/or evaluating the latter.

The network can comprise at least two distinct network nodes that can be connected to the body of the patient. Here, network nodes should be understood to mean assemblies that, as will be explained in more detail below, can communicate with one another and can interchange data and/or commands. The network preferably comprises more than two of such network nodes, for example three, four, or more of such network nodes.

The term, “can be connected to the body of the patient,” can mean a property that allows the arrangement of the network nodes on the body, in the body, in the direct vicinity of the body or combinations thereof, such that signals can be coupled into and/or decoupled out of the body, for example, the body tissue and/or the bodily fluid. In the process, an intra-body conduction mechanism can be used as the basis for communication for the data transmission. By way of example, the network nodes may have appropriate electrodes that can be used for coupling-in and/or decoupling the signals. By way of example, provision can be made for one or more electrode faces, which can be brought into direct or indirect contact with the skin surface of the patient for coupling-in and/or decoupling purposes.

In one embodiment, at least two of the network nodes each have at least one medical function. In another embodiment, three, four or more network nodes each have a medical function. In yet another embodiment, all the network nodes have a medical function. A medical function can mean any pharmaceutical, diagnostic, analytic, therapeutic, surgical, medicinal, or regulatory function or a combination of the aforementioned and/or other combinations, which interact directly or indirectly with bodily functions of the patient. This medical function can be a diagnostic function and/or a medication function.

The network nodes can designed be to communicate directly with one another via the body of the patient and to interchange data and/or commands. A direct communication can mean a communication that does not necessarily need access to an external central communication instrument arranged outside of the body of the patient. In one embodiment, the network nodes together can form a near-field network within the body. Over this network, the network nodes can communicate with one another and interchange data and/or commands. For example, as a result of being integrated into corresponding network nodes, various instruments and/or sensors used on and/or in the body can be connected to form a network, which instruments and/or sensors can first interchange data and, secondly, can also assume a control, for example they can control implanted instruments, as a result of this and/or other data. The data and/or commands can be respectively interchanged via the body such that a near-field intra-body network can be generated.

At least one of the network nodes can comprise a sensor, that is an element for qualitative and/or quantitative acquisition of at least one measurement variable, such as, for example, a physical and/or chemical measurement variable. At least one of the network nodes can comprise at least one of the following sensors: a sensor for registering at least one analyte in a bodily fluid such as, for example, glucose, lactate, CO₂, Hb, Hb-O₂; a sensor for registering at least one bodily function, such as, for example, a kidney function; a blood-pressure sensor; an oximeter; a heart-rate monitor; a motion detector; a temperature sensor or combinations thereof. However, any suitable type of sensor or combination of types of sensors can be used.

In one embodiment, at least one of the network nodes can also comprise at least one sensor that can wholly, or partly, be implanted into the body of the patient. This sensor can register at least one measurement variable in a body tissue and/or a bodily fluid, such as, for example, a concentration of at least one analyte. Glucose and lactate are examples of such an analyte.

In another embodiment, at least one of the network nodes can comprise at least one actuator, that is, an element that emits at least one signal and/or causes at least one effect in another element and/or in the body. For example, one or more of the following actuators may be: an actuator for influencing at least one bodily function such as, for example, an electrical actuator and/or a mechanical actuator; a valve such as, for example, a valve for urinary control; and/or a medication actuator such as, for example, a medication pump. The at least one actuator can allow for targeted direct, or indirect, influencing of bodily functions and/or controlling of other elements.

Furthermore, the network, or at least one of the network nodes, may comprise at least one data storage medium such as, for example, a volatile and/or nonvolatile data storage medium. The network node can undertake data acquisition of data from this network node, for example from a sensor of this network node, and/or from other network nodes.

In one embodiment, a connection with at least one sensor for continuous monitoring (“continuous monitoring sensor”); a medication pump such as, for example, an insulin pump; and a monitoring system based on “near-field intra-body communication” can be implemented. The network nodes can then form the near-field intra-body network.

The at least one sensor can, for example, be partly, or wholly, implanted and can, for example, collect measurement data continuously, or discontinuously, at brief intervals. By way of example, measurement data relating to glucose in an interstitium and/or in whole blood (for example, from veins or arteries) can be collected. This data overall can be collected, possibly processed, and possibly converted into a suitable format, can be actively transmitted to the appropriate addressees in the network, and can be recalled from there, for example, on request.

The glucose sensor may, alternatively or in addition thereto, be replaced and/or complemented by sensors of other types in order to measure other physical parameters, such as blood pressure, heart rate, temperature, or combinations thereof and/or other parameters. Alternatively, or in addition thereto, it can also be possible to measure chemical parameters, for example, blood, oxygen, and/or further analytes. In one embodiment, the sensors can register all specific data, optionally already process into a suitable format in situ, and then, likewise optionally, buffer the data into a format suitable for communication. By way of example, the format can be suitable for an asynchronous communication.

In one embodiment, the network may be designed such that at least one network node can control network. In another embodiment, at least two network nodes can control network. In yet another embodiment, all the network nodes can control network. By way of example, this control can be a “master” of the system. For example, one network node, a plurality thereof, or all network nodes can be configurable as the “master” and, thereby, can be charged with coordinating the entire network. In one embodiment, a network node, that can comprise an indication function, can assume this master role. A network node that has a different type of human-machine interface such as, for example, appropriate input/output, can also be used. Such input/output can be, for example, at least one vibrator and/or an acoustic transducer.

The network may also assume control and/or regulating roles in a closed fashion, that is, without the need for external intervention. By way of example, at least one of the network nodes may comprise at least one sensor for registering at least one measurement variable of the patient. The at least one other network node can then comprise at least one therapeutic device such as, for example, a medication device. The network can control and/or regulate the therapeutic device in accordance with the measurement variable via the body of the patient, that is over the intra-body network. The control function and/or the regulation function may be assumed by one or more of the network nodes. By way of example, the network node can also comprise the sensor and/or the network node comprising the therapeutic device. The control and/or regulation functions may also be distributed. It can also be possible to have a specific communication profiles between the network nodes. By way of example, such specific communication profiles may be between a glucose sensor and an insulin pump. This can also create a closed-loop control system. Overall, this closed regulation and/or control function within the network can take place such that it does not require external intervention by the patient and that the patient does not always have to be informed about these processes. By way of example, the patient may be aware of only results, status information, or acute alarms.

In one embodiment, the network nodes can communicate with each other over asynchronous data transmission. The control and coordination of an intra-body network can assume a protocol that is matched specifically to the requirements of such an intra-body network. In the case of asynchronous data transmission such as, for example, the ITU 34.13 standard, the bytes to be transmitted can be transmitted asynchronously, that is, generally at any time. Generally, there can be approximate synchronization between the respective transmitter and the receiver for only the duration of one byte. Since the synchronization quality requirements between transmitter and receiver are lower, the synchronization can be reached more quickly.

In general, freely available wireless frequencies can be filled with standard applications within a very short time frame. Accordingly, problems may occur in medical applications where time is critical, particularly in respect to real-time data communication. The present disclosure can be able to reduce drastically such time conflicts and can allow increasingly complex data communication and data structures in the vicinity of the body. In addition, as a result of the low extracorporeal interference potential during the communication, such systems can also be used in critical regions, such as, for example, an emergency room, an intensive care unit, in explosion-proof surroundings, or any other similar region.

At least one of the network nodes can also carry out a failsafe function. Such a failsafe function can mean independent identification of abnormal states and, if need be, resulting in an appropriate reaction to these abnormal states. In one embodiment, there can be a plausibility check of transmitted data, commands, or measurement values. By way of example, if agreed upon error discrimination thresholds are exceeded, or if regions that are considered “normal” are departed from, a conclusion can be drawn that an abnormal state or error is present. Direct measures can then be initiated. By way of example, the network node can carry out at least one error routine if an error state is identified. By way of example, such an error routine may involve switching off the supply voltage in order to protect against biologically critical dangers. Other error routines can also possible. Alternatively or in addition thereto, at least one network node can emit an alarm to the patient, particularly if there is a malfunction of the network and/or if abnormal bodily functions occur.

In one embodiment, at least one of the network nodes can comprise an indication device. In one embodiment, this indication device can comprise an indication device that can be worn on a wrist of the patient. In one exemplary embodiment, the indication device can be integrated into a wrist watch. In this embodiment, the wrist can act as an interface between the network node with the indication device and the body of the patient because the wrist watch can establish direct contact, such as, for example, by suitable electrodes. As an alternative to an indication device, or in addition thereto, at least one network node can comprise at least one other input and/or output device such that the patient and/or medical practitioner can have direct access to the network.

One or more further interfaces can be included in order, for example, to communicate with other components not included in the network. This interface can, for example, comprise a wired and/or a wireless interface. In one embodiment, at least one of the network nodes can additionally communicate outside of the body, such as, for example, for far-field communication. Thus, one or more of the network nodes can comprise far-field communication such that they can transfer information to communication networks such as, for example, BlueTooth, WLAN, GSM, and/or computer networks. In one exemplary embodiment, data can continue to be collected, compressed and negotiated. Such data transfer out of the network can be an upload. Instructions and data can also be transmitted to the network nodes, that is, into the network as a download. In one embodiment, the at least one far-field communication node can be situated in an indication instrument on the body surface. In general, communication nodes can be fully implanted and/or arranged on the body surface and/or also arranged at a distance from the body surface.

A further aspect of the present disclosure comprises an energy supply for an individual network node, a plurality of network nodes, or of the entire network. Unlike RFID technology, the present network, which, for example, can work over a capacitive coupling to the body, can comprise at least one separate energy supply as a result of the generally low energy coupling. This energy supply can be the form of integrated, primary batteries and/or secondary batteries and/or other types of electrical energy reservoirs. This can require actions by the wearer at regular or irregular intervals, particularly in respect of replacing and/or recharging the electrical energy reservoirs. Additionally, this can require complex interventions, particularly in the case of implants. Recharging can take place in a non-invasive fashion by means of a contact and/or by applying an external alternating magnetic field.

Alternatively, or in addition thereto, the network can use the energy source of the body and/or the surroundings of the network as an energy source. Accordingly, at least one of the network nodes may extract energy from the body and/or surroundings of the body and to use this energy to supply the network node and/or other network nodes, or the entire network with energy. In the process, the energy can be extracted from the body and/or the surroundings of the body in a number of different ways. By way of example, thermal energy can be extracted. Vibrational energy can also be extract, for example by piezoelectric generators.

Alternatively, or in addition thereto, the corresponding network node can also use an electrochemical energy source. By way of example, electrochemical energy may be extracted from the glucose surrounding an implanted sensor. In one embodiment, this energy can be extracted such that it does not, under any circumstances, influence the measurement value of the glucose such as, for example, by depleting the glucose in the vicinity of the measurement site. The network node can for example comprise at least one electrochemical sensor. The electrochemical sensor can register at least one measurement variable in a sensor mode and can extract energy by electrochemically in at least one energy extraction mode. By way of example, it can be possible to switch between the two modes of the sensor. However, to a certain extent, both modes can also be carried out simultaneously and/or there can be some temporal overlap.

High field strengths and the possible resulting dangerous voltages should be avoided wherever possible in the network. Hence, the protective extra-low voltages should be less than 42 V if possible. In order to have a good signal-to-noise ratio, specific, secure communication protocols can be used because the data transfer rate can generally be low in the case of body networks. This can allow a large proportion of the available bandwidth to be used for redundancy and hence for data safety. This consideration can also take account that a proposed network should not interfere with diagnostic and/or therapeutic apparatuses, even in the case of an intensive-invasive intervention in the body.

The network, in general, can have a flexible design because the medical functions of the network can generally be matched individually to individual patients. Thus, individual network nodes can be removed from the system as desired and/or can be added to complement the system. The network can identify new networks, for example, automatically, and link these into the network.

In one embodiment, the network can also comprise at least one portable hand-held instrument, with at least one indication function, that can be integrated into the network and can also be decoupled from the network. In one embodiment, this portable hand-held instrument can be a medical measurement instrument such as, for example, a blood-glucose measurement instrument. Alternatively, or in addition thereto, the hand-held instrument can also comprise at least one cellular telephone, that is, an instrument designed for mobile data transmission. The network can accordingly link automatically the hand-held instrument into the network when the hand-held instrument makes contact with the body of the patient. In one embodiment, the contact can be with a hand of the patient, that is, a contact point in which signal-coupling into the body and/or signal-decoupling from the body can be enabled.

Active temporary linking of a network into a medical system such as, for example a surgical system, an intensive-care medical system, an anesthetics system or combinations thereof, can be possible. The medical system can comprise at least one communication device that can detect, for example automatically, the presence of a network such as, for example, a “body area network” (BAN). The communication device can further communicate with the network and link into the medical system. In one embodiment, this communication can be over far-field communication. The medical system can interchange data and/or commands with the network.

In addition, the network node can be used use in a network. The network node can comprise at least one medical function such as, for example, a diagnostic function and/or a medication function. The network node furthermore can comprise at least one communication unit, which can be connected to the body of the patient and can communicate directly with other network nodes of the network via the body of the patient and can interchange data and/or commands.

The network, the network node, and the medical system can have a number of advantages over similar known devices. For example, the new diagnostic methods can be implemented by simplified networking of relevant intra-body parameters (within the BAN) and extracorporeal parameters (for example, within a far field) by the network nodes. A more precise diagnostic statement and/or possibly an improved therapy can be possible by a permanent networking of the parameters. Furthermore, the network can be adapted to the personal patient situation with little complexity. By way of example, this can take place in respect to the parameters to be used, in respect to the spatial arrangement, and/or in respect to the temporal design of measurements and/or other medical measures. Furthermore, through adapted conduction mechanisms for transmitting the data, the data can be scattered minimally in space. Avoiding unnecessary scattering of the spatial data traffic can lead to both an increase in the personal data security and a reduction in data errors, in particular, as a result of collisions.

It can be possible to simplify steps in initializing, conditioning and calibrating the networks as a result of combining logical and ergonomic actions by the patient with appropriate functional sequences. By way of example, for a glucose measurement in the interstitium for the purpose of a calibration, a whole blood measurement value can automatically be routed from a blood-glucose meter in the hand of the patient to a long-term sensor (“continuous monitoring sensor”).

Specific networking can result in significantly less energy being required for the network nodes than in the free field. As a result, the system overall can become more energy efficient and the handling steps by the patient for acquiring energy can be avoided. This can also allow for a flexible, decentralized energy concept. Since less energy is required for communication, individual network nodes such as, for example a glucose sensor, can extract energy from the direct vicinity of the network node. Furthermore, the network can easily and flexibly be adjusted to the required general framework. By way of example, state profiles can be specifically monitored by simple networking. As a result, comprehensive healthcare management and/or management in competitive sport may be possible.

Since the field strengths can be reduced during the intra-body transmission, the networks can also be operated in critical surroundings such as, for example, in intensive-care units, in an emergency room, in areas prone to explosions (for example, in the surroundings of gas stations), or in an airplane. The intra-body networks can even temporarily act as components of more comprehensive, intensive-care diagnostic systems and hence can, for example, provide support during surgery and/or in anesthetics.

Additional advantages can emerge from the respectively expedient linkage of the individual components of the network and/or of the medical system. These components can respectively be interconnected with the optimum network technique for the various requirements. By way of example, sensors and/or actuators can be interconnected over the BAN, that is, the network, while the entire network and/or individual components of the network can be connected to remaining components of the medical system over for example, mobile radio and/or other types of far-field communication. Furthermore, far-field frequency bands generally can have a capacity problem or will have such a capacity problem in the near future.

Self-learning organizing networks can be feasible using the network. By way of example, a network node can be associated with a user after the user touches the network node. After attaching the network node, the network nodes can then communicate with one another and, for example, can interchange modalities for the further cooperation in the network.

The aforementioned optional failsafe concepts can likewise take into account the network. Thus, for example, individual network nodes can make independent decisions and can carry out measures in defined situations. By way of example, a “continuous monitoring sensor” can determine the discharge of substances such as, for example, a discharge of electrode material and/or other sensor components. By way of example, a discharge of copper out of an electrode and/or a feed line can be possible. If a discharge is determined, it can be possible, for example, to initiate corresponding measures such as, for example, a current interruption. It may also be possible to organize failsafe strategies with further network nodes such as, for example, in a self-organizing fashion. This can allow the failsafe modality to be extended to the entire network. Hence, a plurality of network nodes can be involved in the at least one failsafe function.

FIG. 1 illustrates a schematic diagram of signal transmission from a transmitter 112 to a receiver 114 via a body 110. Transmitter 112 and receiver 114 can each comprise electrodes 116, which can be applied directly to a skin surface 118 or can be arranged in the direct vicinity of the skin surface 118. Both transmitter 112 and receiver 114 can each comprise an energy source 120. The energy source 120 can, for example, comprise at least one energy reservoir such as, for example, a battery, a rechargeable battery, an energy generator or combinations thereof. In the transmitter 112, this energy source 120 can feed a signal generator 122, which can actuate the electrodes 116 of the transmitter 112, for example, with an AC voltage. This can produce an electric field 124 in the body 110, which can be used for near-field intra-body transmission. In addition to the energy source 120 and the electrodes 116, the receiver 114 can additionally have, for example, one or more amplifiers 123 for amplifying signals recorded by the electrodes 116 and, optionally, for completely, or partly, processing the signals. Furthermore, the transmitter 112 and receiver 114 can comprise additional components (not shown) such as, for example, data-processing instruments, instruments for signal processing or combinations thereof.

The principle of intra-body data transmission is known from the prior art. The principles and methods of intra-body data transmission can also be utilized within the scope of the present disclosure such as, for example the principles relating to the coupling-in and/or decoupling of signals and/or the processing of signals. FIG. 1 illustrates the fundamental principle of a non-ground-related near-field intra-body communication, which is shown in an exemplary embodiment as a bipolar point-to-point connection between transmitter 112 and receiver 114. However, alternatively, or in addition thereto, ground-related near-field intra-body communications can also be possible. More complex embodiments are also possible. Thus, any transmitter-receiver nodes can be attached to the body 110. The transmitters 112 can also act as receivers 114 and vice versa.

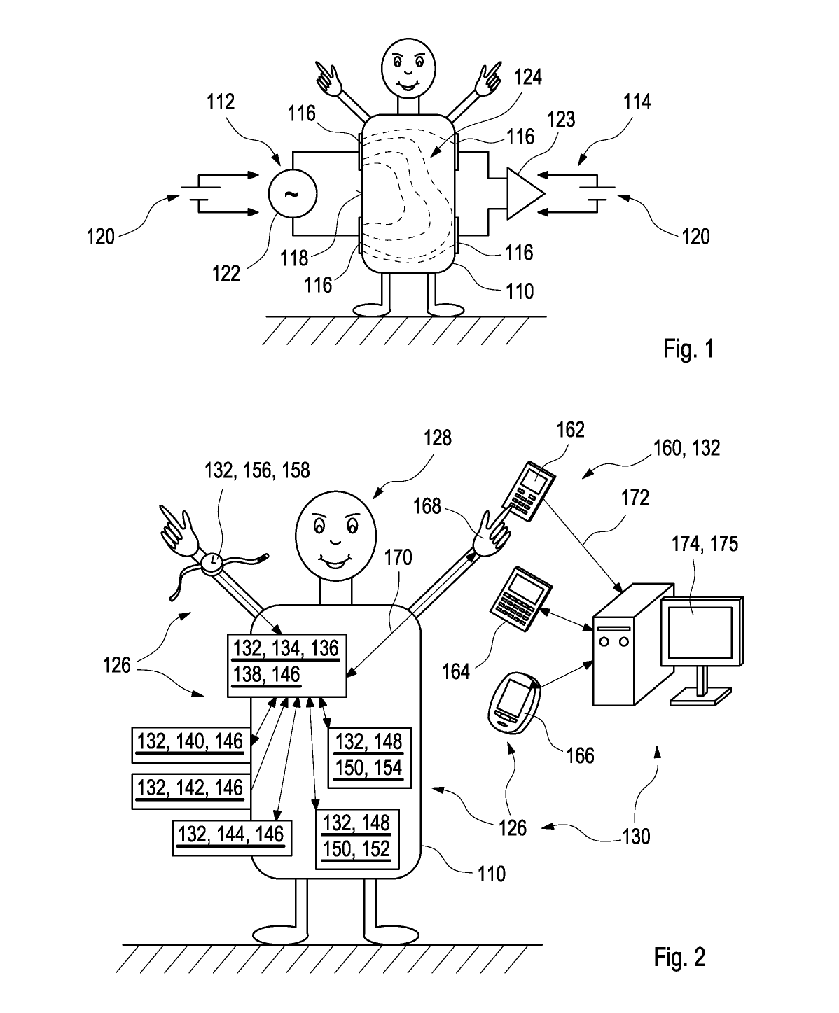

FIG. 2 illustrates an exemplary embodiment of a network 126 for monitoring bodily functions of a body 110 of a patient 128 and an exemplary embodiment of a medical system 130 into which the network 126 can be linked. A network 126, which can be used in the field of diabetes care, is illustrated as an example. It can also be possible to monitor other types of bodily functions of a patient. It can also be possible to monitor other types of clinical pictures and/or other types of health states. In addition, the term patient 128 could mean, in general, any human or animal, without being restricted to users with abnormal body functions.

The network 126 can comprise a plurality of network nodes 132. In one exemplary embodiment, the network 126 can be a star-shaped network and can comprise a network node 132 with a glucose sensor 136 as a central network node 132, which can also act as a master network node 134. In one exemplary embodiment, this glucose sensor 136 can be an implantable sensor 138. The implantable sensor 138 can be a long-term sensor, or a “continuous monitoring sensor,” which can, at least in part, be implanted into body tissue of the patient 128. In one embodiment, the master network node 134 can comprises at least one transmitter 112 and at least one receiver 114 in addition to the glucose sensor 136. The transmitter 112 and receiver 114 can also, at least in part, have an identical component design. In one embodiment, all other network nodes 132 can comprises at least one transmitter 112 and at least one receiver 114. By way of example, one, two, or more electrodes 116 can be provided in an analogous fashion as to the schematic diagram in FIG. 1.

In addition to the master network node 134, the network 126 can comprise a plurality of additional network nodes 132, which, optionally, can also be replaceable. The additional network nodes 132 can be a temperature sensor 140 such as, for example, an infrared temperature sensor, a skin-contact temperature sensor, an implanted and/or implantable temperature sensor or the like. Furthermore, the network 126 can, for example, comprise one or more blood-pressure sensors 142, analyte sensors 144, or other suitable type of sensors. The sensors have been generically denoted by the reference sign 146 in FIG. 2.

As an alternative or in addition to sensors 146, the network nodes 132 can also comprise other types of medical functions, for example actuators 148 that can be used in a medical context. By way of example, provision can be made for a network node 132 with a medication device 150 in the form of an insulin pump 152. Alternatively or in addition thereto, provision can also be made for other types of medication devices 150, which can also be generically described as “drug-delivery” systems 154.

In one exemplary embodiment of the network 126 illustrated FIG. 2, the network 126 can comprise an indication device 156. In one embodiment, the indication device can be in a wrist watch 158, which can be integrated into the network 126. By way of example, the wrist watch 158 can have an appropriate program-technical setup, The wrist watch 158, as a network node 132, can comprise electrodes 116 and transmitters 112 and/or receivers 114, and, optionally, can also comprise further apparatuses such as, for example, at least one signal generator 122 and/or at least one amplifier 123. Hence, the wrist watch 158 can serve as visual interface between the patient 128 and the network 126. Moreover, the wrist watch 158 can also be used as a network node 132 with input functions, which, for example, can allow the patient 128 to enter commands, to control the network 126, and/or to query information from the network 126.

The network 126 illustrated in FIG. 2 can optionally comprise further network nodes 132 with an indication function and/or input and output. For example, one or more hand-held instruments 160 may be linked in as network nodes 132. The hand-held instruments 160 can comprise one or more cellular telephones 162, portable computers 164 (for example, personal digital assistants, PDAs), or portable measurement instruments 166 such as, for example, blood-glucose measurement instruments. By way of example, the hand-held instruments 160 can be linked into the network 126 via hand 168 of the patient 128 in order to interchange, for example, calibration data 170 or the like with the remaining network nodes 132. Control commands, measurement data, or the like can also be interchanged.

As illustrated in FIG. 2, the network 126 can also be linked into a medical system 130 such as, for example, into a healthcare system. The network 126 can also automatically switch itself into for the support one or more healthcare systems such as, for example, in the case of an emergency diagnosis during an intervention by an emergency doctor, in an ambulance, during anesthesia, during surgery, or in any other suitable similar situations. One advantage in using the network 126 in this case can be the fact that, for example, the sensors 146 and/or other components of the network 126 do not have to be applied, but are already at least partly present on the patient. The medical system 130 can, for example, interchange measurement data, information, control commands, or the like with the network 126 over a data connection 172. By way of example, far-field communication can be used, for example over a cellular telephone 162 of the network 126. By way of example, the medical system 130 can comprise one or more computers 174 and/or computer networks, as illustrated in FIG. 2. The medical system 130 can furthermore comprise one or more communication devices 175, which can also be components of the computer 174 and/or the computer network. By way of example, at least one communication device 175 can be establish and can maintain the data connection 172 to the network 126.

The embodiment of depicted in FIG. 2 is only one example. The network 126, the networks nodes 132 and associated functions can also include other embodiments. For example, in another exemplary embodiment, one or more interstitial glucose sensors can be partly, or wholly, implanted into a human or animal body 110. Alternatively, or in addition thereto, further analyte sensors 144 can likewise be implanted. Additional physical sensors 146 can be used outside of the body such as, for example, a blood-pressure sensor 142, an oximeter, a heart-rate monitor, or any other suitable sensor.

Alternatively, or in addition thereto, further physical and/or chemical parameters can be registered by the sensors 146 for a body status, particularly in the case of patients 128 in a critical overall state. Thus, it can be possible to measure, for example, lactate, CO₂, Hb, Hb-O₂, kidney parameters (particularly in the context of multiple organ failure), urinary functions, or combinations of the aforementioned and/or other parameters. Moreover, the sensors 146 can, additionally or alternatively, for example, comprise motion detectors. In addition to actuators that can be used in a medication device 150 (for example, dosage actuators), different types of actuators can, additionally or alternatively, also be used as actuators 148 such as, for example valves, for example for urinary control.

Furthermore, actuators 148 can, for example, be used in the insulin pump 152 and/or in other types of medication device 150. The insulin pump 152 can, for example, be arranged outside of the body, for example, with an implantable catheter. Alternatively, or in addition, use can be made of other types of “drug-delivery” systems 154, which can optionally likewise comprise one or more actuators 146.

The wrist watch 158 with the indication device 156 can act as a permanent display, for example, for indicating a status or for indicating an alarm. By way of example, the indication device 156 can allow optical and/or acoustic output of information. Alternatively, or in addition thereto, additional instruments can also be linked into the network 126, particularly sporadically; these instruments are indicated in FIG. 2 by the hand-held instruments 160. In addition to the cellular telephone and the portable computer 164, portable measurement instruments 116 can be incorporated such as, for example, blood-glucose measurement instruments, blood-pressure measurement instruments, or the like. In general, these hand-held instruments 160 can be picked up by the hand 168 of the patient and hence can be linked-in as part of the network 126, at least on a temporary basis. Electrodes 116, suitable for the “near field intra-body communication,” can, for example, be on these hand-held instruments 160. Such temporary network nodes 132 with hand-held instruments 160 can control, initialize and/or calibrate further components of the network 126. However, in general, the term “hand-held instrument” does not necessarily restrict such instruments to portable instruments. In general, these are instruments can also have a stationary design and can establish a contact with a hand 168 of the patient.

In FIG. 2, a spot-blood-glucose measurement instrument can, for example, be used as a portable measurement instrument 166. By way of example, when hand contact is made, the measurement instrument 166 can, as a basis for a calibration, transmit a blood-glucose value, measured in real-time, directly to the continuous measurement system of the glucose sensor 136 with the implantable sensor 138 measuring glucose in the interstitium of the patient 128. By way of example, this can be a precondition for an artificial pancreas.

Furthermore, it can be feasible for whole-blood measurement systems to be used as glucose sensor 136 and/or as portable measurement instrument 166 and/or in further network nodes 132. By way of example, these systems can be equipped with devices for extracting blood by minimally invasive methods and/or for direct measurement. By way of example, such measurement systems can then transfer the time at which blood was extracted and/or the time at which the measurement took place to various network nodes 132.

In addition to being linked into the network 126, one or more of the network nodes 132 can communicate outside of the network 126 such as, for example, over a data connection 172. In addition to a wired data connection, wireless transmission techniques can also be used such as, for example, all known transmission techniques. In one embodiment, a far-field transmission can be used. Thus, for example, network nodes 132 that are connected to the hand 168 can assume such transmission functions. By way of example, the hand-held instruments 160, for example the cellular telephone 162, can establish a bidirectional connection in the far field. Alternatively, or in addition thereto, the wrist watch 158 can be suitable for this purpose.

Furthermore, a star-shaped communication structure of the network 126 is illustrated as an example in FIG. 2. In the process, for example, the glucose sensor 136, which can for example be embodied as a glucose patch with an implantable sensor 138, can assume the role of the “master”. However, other network nodes 132 can alternatively, or in addition thereto, assume this role. The role of the master can be assumed by the respective component on a permanent or on a temporary basis. Furthermore, it can also be possible to use communication structures other than the aforementioned star-shaped structure.

By way of example, the master network node 134 can coordinate the communication traffic and can moreover optionally have the role of linking multivariate parameters and, optionally, of generating instructions for other network nodes 132, for example for the actuators 148. Self-learning software structures can also be feasible. Other network nodes 132 can also assume this role. By way of example, structures are possible in which the network 126 is self-organizing. In this example, the best-suited network node 132 can assume the role of the master network node 134, for example on a permanent or a temporary basis.

The communication 126 can take place on asynchronous networks. Each network node 132 can for example have a specific address, over which the network node 132 can be addressed. Data transmission can take place in a packet-oriented fashion. In the process, a message can be decomposed into packets and put into temporal sequence by packet number in the respective receiver. In the case of interference in individual packets, these packets can be sent repeatedly until one or more checking mechanisms, for example a so-called CRC-check, considers the transmission to be accurate.

Since the assumption can generally be made that the amount of energy transmitted is very low and that the noise-to-signal ratio is comparatively bad, it may optionally be possible to develop novel protocols with high redundancy. This can be possible because the information density between the network nodes 132 will generally be comparatively low, and so a high bandwidth can be used for increased redundancy and/or for a low latency time.

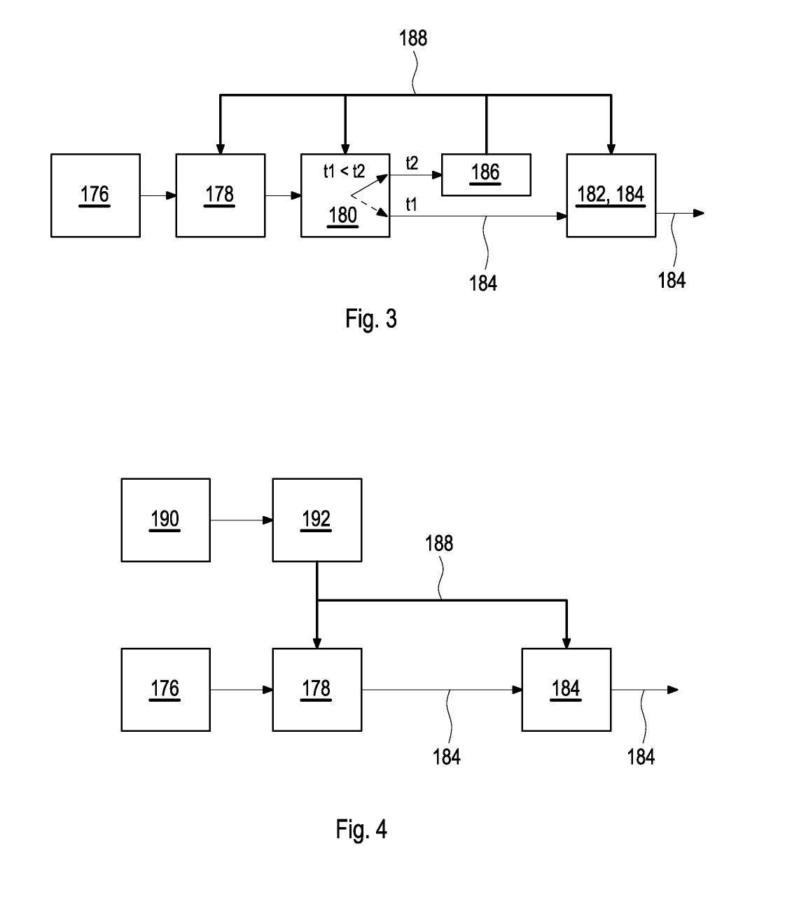

A problem in typical medical networks, such as the networks 126 illustrated in FIG. 2, generally relates to the energy supply of the entire network 126 and/or individual network nodes 132 of the network 126. FIGS. 3 and 4 show different schematic exemplary embodiments of a possible energy supply that can be used in one network node 132, in a number of network nodes 132, or in all network nodes 132. Here, “energy harvesting”, that is, extracting energy, in the surroundings of the glucose sensor 136 is shown as an example. However, fundamentally the principles can also be applied to other types of network nodes 132 and/or to other types of functions. FIG. 3 shows a basic layout for extracting energy, where the same source is used to extract a signal for a sensor 146 and energy for operating the network node 132 and/or individual components of the network node 132 and/or other components of the network 126. By contrast, FIG. 4 shows an exemplary embodiment in which energy is extracted from a separate source.

When energy is extracted from the same source as illustrated in FIG. 3, biochemical system 176 can be used. By way of example, this can be a biochemical redox system, which generates charge and/or current. By way of example, this can be an electrochemical system that is usually utilized in blood-glucose sensors, based on oxidation of blood glucose, and optionally uses enzymes and/or auxiliary materials.

The background for extracting energy as illustrated in FIG. 3 is that such a biochemical system 176 requires comparatively little energy for the measurement, that is the actual measurement rate of the sensor 146. By way of example, typically only 1/1000 of the continuously flowing charge is required for the measurement. The remainder generally is discharged and converted into heat so that the charge does not build up at the measurement site of the sensor 146. However, this component that is generally discharged can also be collected for extracting energy, as indicated in FIG. 3.

Thus, by way of example, the exemplary embodiment as per FIG. 3 can optionally comprise a transducer 178, for example a transducer with low-voltage start, connected to the biochemical system 176. The transducer 178 can be used to extract energy. A switch 180 can be connected to the transducer 178 and can switch between two modes: at least one measurement variable of the sensor 146 can be registered in a sensor mode 182, for example a current and/or a voltage. The at least one measurement variable can be transmitted as a signal indicated in FIG. 3 by reference sign 184. Various embodiments are feasible. The signal can be transmitted 184 to further components of the network node 132 and/or to external components.

By contrast, in a further mode, which is symbolically referred to as energy extraction mode 186 in FIG. 3, the excess charge, the excess current, or the unutilized voltage can be utilized to extract energy. As a result of switching between the modes, the energy extraction in this example under no circumstances influences the measurement value, for example, as a result of depleting the glucose in the vicinity of the measurement site. By way of example, this can afford the possibility of producing and providing energy for the sensor 146, the network node 132, and/or further components of the network 126. In FIG. 3, this is indicated symbolically by the provision arrow 188 indicating that the transducer 178 and/or the switch 180 and/or the signal transmission 184 can be provided with electrical energy. The reference sign 186 for the energy extraction mode in FIG. 3 is merely exemplary. The block denoted by the reference sign 186 in FIG. 3 can also comprise technical elements that can be connected to the energy extraction mode. Thus, the energy extraction mode 186 can also comprise a conversion of energy and/or at least one energy reservoir.

Switching between the two modes can for example, as indicated in FIG. 3, be controlled in a temporal fashion by the times t₁ and t₂. Other switching methods are also feasible. That is to say in addition to time-controlled, for example clocked, methods, temporally flexible methods, which can, for example, specifically react to a measurement query, are also feasible. Overall, the method per FIG. 3 can for example generate approximately 1 μWs of energy in the case of a sensor 146 that can be implemented. Accordingly, as a result of the scarce energy resources, energy-saving applications can be preferred for the electronics.

By contrast, FIG. 4 shows a concept in which the energy is extracted from a separate source. By way of example, provision can once again be made for a biochemical system 176, for example in a sensor 146. However, other types of sensors 146 and/or actuators 148 can also be used. Furthermore, provision can once again made for a measurement value transducer 178, and also an appropriate signal transmission 184.

However, in contrast to the embodiment as per FIG. 3, there is separate energy extraction in FIG. 4. Accordingly, provision can be made for an energy extraction device 190, which can draw energy from the body 110 and/or surroundings of the body 110. By way of example, movement energy can be generated by piezoelectric elements, thermal energy may be generated from temperature differences, or similar methods may be used. By way of example, this extracted energy can be temporarily stored in an energy reservoir 192 and can then be provided to further system components. The provision is denoted by the reference sign 188. In the exemplary embodiment illustrated in FIG. 4, the transducer 178 and the signal transmission 184 can be fed with electrical energy in an exemplary fashion.

The idea of separate energy extraction illustrated in FIG. 4 can be advantageous over the energy extraction illustrated in FIG. 3 in that parallel energy extraction generally can lead to higher and more independent energy withdrawal. By contrast, in the design in FIG. 3, a noise problem may occur as the energy consumption of the processing electronics reduces; however, this noise problem can likewise be reduced by appropriate measures such as, for example by integrating the signal. The parallel extraction of energy in FIG. 4 generally does not require any such additional measures.

The network 126 can also comprise one or more additional energy reservoirs 192. By way of example, the energy reservoir 192 can be one or more batteries, rechargeable batteries, supercapacitors, or the like. Provisions can also be made for rechargeable and/or non-rechargeable energy reservoirs 192.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

For the purposes of describing and defining the present disclosure, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A network for monitoring bodily functions of a patient, the network comprising:
at least two network nodes connected to a body of the patient, wherein each of the at least two network nodes have at least one medical function, wherein the at least two network nodes communicate directly with one another via the body of the patient and interchange data and/or commands and wherein the at least two network nodes control the network.

2. The network according to claim 1, wherein at least one of the at least two network nodes comprises at least one of the following sensors: a sensor for registering at least one analyte in a bodily fluid; a sensor for registering at least one bodily function; a blood-pressure sensor; an oximeter; a heart-rate monitor; a motion detector; a temperature sensor; or combinations thereof.

3. The network according to claim 2, wherein the at least one analyte in a bodily fluid is glucose, lactate, CO₂, Hb, Hb-O₂ or combinations thereof.

4. The network according to claim 2, wherein the at least one bodily function is kidney function.

5. The network according to claim 1, wherein at least one of the at least two network nodes comprises at least one sensor that is wholly or partly be implanted into the body of the patient, wherein the implantable sensor registers at least one measurement variable in a body tissue and/or a bodily fluid.

6. The network according to claim 5, wherein the at least one measurement variable is a concentration of at least one analyte.

7. The network according to claim 1, wherein at least one of the at least two network nodes comprises at least one actuator, wherein the at least one actuator comprises an actuator for influencing at least one bodily function.

8. The network according to claim 7, wherein the actuator comprises an electrical actuator, a mechanical actuator, a valve, a medication actuator, or combinations thereof.

9. The network according to claim 8, wherein the valve is a valve for urinary control.

10. The network according to claim 8, wherein the medication actuator is a medication pump.

11. The network according to claim 1, wherein at least one of the at least two network nodes comprises at least one sensor for registering at least one measurement variable and wherein at least one of the other network nodes comprises at least one therapeutic device, wherein the network controls and/or regulates the therapeutic device according to the at least one measurement variable via the body of the patient.

12. The network according to claim 11, wherein the at least one therapeutic device is a medication device.

13. The network according to claim 1, wherein all network nodes control the network.

14. The network according to claim 1, wherein the network nodes communicate over asynchronous data transmission.

15. The network according to claim 1, wherein at least one of the at least two network nodes comprises an indication device.

16. The network according to claim 15, wherein the indication device can be worn on a wrist of a patient.

17. The network according to claim 15, wherein the indication device is integrated into a wrist watch.

18. The network according to claim 1, wherein at least one of the at least two network nodes carries out a failsafe function and carries out at least one error routine if an error state is identified.

19. The network according to claim 1, wherein at least one of the at least two network nodes emits an alarm to the patient if there is a malfunction of the network and/or if abnormal bodily functions occur.

20. The network according to claim 1, wherein at least one of the at least two network nodes communicates outside of the body.

21. The network according to claim 20, wherein the communication is far-field communication.

22. The network according to claim 20, wherein the communication is for uploading and/or downloading data.

23. The network according to claim 1, wherein at least one of the at least two network nodes extracts energy from the body and/or from surroundings of the body and uses this energy to supply the network node and/or other network nodes with energy.

24. The network according to claim 23, wherein the least one network node comprises at least one electrochemical sensor, wherein the electrochemical sensor registers at least one measurement variable in a sensor mode and wherein the electrochemical sensor extracts energy by electrochemical means in an energy-extraction mode.

25. The network according to claim 1, further comprising, at least one portable hand-held instrument, wherein the portable hand-held instrument has at least one indication function and is integrated into the network and is decoupled from the network, wherein the network automatically links the portable hand-held instrument into the network when the hand-held instrument makes contact with the body of the patient.

26. The network according to claim 25, wherein the at least one portable hand-held instrument comprises a medical measurement instrument, a cellular telephone or combinations thereof.

27. The network according to claim 25, wherein the network automatically links the portable hand-held instrument into the network when the hand-held instrument makes contact with a hand of the patient.

28. A network node having at least one medical function for use in a network as claimed in claim 1, the network node comprising:
at least one communication unit connected to the body of the patient which communicates directly with other network nodes of the network via the body of the patient and interchanges data and/or commands, wherein at least two of the network nodes control network.

29. The network node according to claim 28, wherein the at least one medical function comprises a diagnostic function, a medication function or combinations thereof.

30. A medical system, the medical system comprising:
at least one communication device, wherein the communication device detects the presence of the network as claimed in claim 1 and communicates with the network, wherein the medical system interchanges data and/or commands with the network.

31. The medical system according to claim 30, wherein the medical system comprises a surgical system, an intensive care medical system or combinations thereof.

32. The medical system according to claim 30, wherein the communication device automatically detects the presence of the network.

33. The medical system according to claim 30, wherein the communication device communicates with the network over far-field communication.
